(19) United States
(12) Plant Patent Application Publication
(10) Pub. No.: US 2016/0165780 P1
(43) Pub. Date: Jun. 9, 2016

(54) Stevia plant named 'SW 201'

(50) ;

(71) Applicant: S&W Seed Company, Five Points, CA (US)

(72) Inventors: Clinton C. Shock, Ontario, OR (US); Cheryl A. Parris, Ontario, OR (US)

(73) Assignee: S&W Seed Company, Five Points, CA (US)

(21) Appl. No.: 14/544,200

(22) Filed: Dec. 8, 2014

(51) Int. Cl. A01H 5/00

(52) U.S. Cl. PLT/258

(57) ABSTRACT

A new and distinct Stevia rebaudiana plant named ‘SW 201’ is described. The new variety is distinguished from other Stevia varieties by excellent sweet leaf taste with very low bitterness and aftertaste, superior overwintering capability, high plant vigor, high leaf yield, late flowering, and high steviol glycoside levels.

### GENUS AND SPECIES

Stevia rebaudiana.

### VARIETY DENOMINATION

SW 201

### BACKGROUND OF THE INVENTION

Stevia rebaudiana is a plant species in the sunflower (Asteraceae) family, which has naturally sweet leaves. The active compounds that impart the sweet flavor to stevia leaves are steviol glycosides. The most common steviol glycosides are stevioside and rebaudioside A, both of which have up to 250 to 400 times the sweetness of sugar. Stevioside tends to be more prevalent in unimproved lines of stevia leaves than rebaudioside A. The development of new varieties of Stevia rebaudiana with higher levels of rebaudioside A than stevioside is desirable for use as a sugar substitute.

The present invention relates to a new and distinct variety of Stevia rebaudiana, the ‘SW 201’ line, which has very sweet leaves with high levels of steviol glycosides and very low bitterness and aftertaste, excellent overwintering, high plant vigor, high leaf yield, and late flowering.

The ‘SW 201’ line was derived in 2012 from a single plant in a population of recurrently selected Stevia rebaudiana, the population of which was originally grown from seed in an outdoor field in Chowchilla, Calif., USA, under natural lighting. The plant that produced the ‘SW 201’ line was selected from its population based upon the following characteristics, which were measured and/or observed between 2012 and 2014.

Leaf Taste:

The leaves of the ‘SW 201’ line were found to have an excellent sweet taste in comparison to a majority of the plant population from which the line was derived and very little bitterness and aftertaste in comparison to most stevia varieties.

Overwintering:

The ‘SW 201’ line was found to have superior overwintering in comparison to the majority of the plant population from which the line was derived, the latter of which died over the course of the winter.

Plant Vigor:

The ‘SW 201’ line was found to have greater growth with higher leaf yield and later flowering (an advantage for leaf production) in comparison to the majority of the plant population from which the line was derived.

Steviol Glycosides:

The leaves of the ‘SW 201’ line were found to have high levels of steviol glycosides (over 18%) with rebaudioside A accounting for over 60% of the total steviol glycoside content in the leaves (see, Table 1).

Upon initial selection, the ‘SW 201’ line was asexually reproduced from rooted cuttings in May 2012. Plant yield and leaf quality of the ‘SW 201’ line were confirmed in April through October 2014 through replicated yield trials in the following three locations: Yuma, Ariz.; Indio, Calif.; and Ontario Oreg. At each of the three locations, the performance of the ‘SW 201’ line grown from cuttings was compared to seedlings grown from bulk commercial seed (“the check variety”). Table 1 compares various characteristics of an average sampling of leaves of the ‘SW 201’ line with an average sampling of leaves of the check variety.

TABLE 1

| | SW 201 | Check Variety |
|---|---|---|
| Percentage of Rebaudioside A | 11.17% | 6.85% |
| Percentage of Stevioside | 5.29% | 6.42% |
| Percentage of total Steviol | 18.04% | 14.71% |
| Glycosides | | |
| Ratio of Rebaudioside | 2.19 | 1.11 |
| A: Stevioside | | |
| Percentage of Rebaudioside A to | 60.9% | 46.8% |
| total Steviol Glycosides | | |

The data in the foregoing table shows that in comparison to the check variety, the ‘SW 201’ line contained more rebaudioside A (11.17% vs. 6.85%); less stevioside (5.29 vs. 6.42%); more total steviol glycosides (18.04% vs. 14.71%); a higher rebaudioside A to stevioside ratio (2.19 vs. 1.11); and a higher percentage of rebaudioside A to total steviol glycosides (60.9% vs. 46.8%). In addition to the foregoing, the ‘SW 201’ line also produced 22% more leaf yield than the check variety with the ‘SW 201’ line producing 4,129 lb of leaf per acre per year versus 3,386 lb of leaf per acre per year for the check variety. Leaf yield results were averaged over three locations with four replications at each location, and two harvest strategies (one or two cuttings per year). The higher yield of the ‘SW 201’ line was due in part to low stand loss from Fusarium and other root diseases that reduced plant stand of the check variety.

### BRIEF SUMMARY OF THE INVENTION

The following characteristics of the new ‘SW 201’ line have been repeatedly observed and can be used to distinguish the ‘SW 201’ line as a new and distinct cultivar of a Stevia plant:

1. Excellent sweet flavor to the leaves with very little bitterness and aftertaste;

2. Excellent overwintering;

3. High plant vigor;

4. High plant yield;

5. Late flowering;

6. Rebaudioside A content of 11.17%;

7. Stevioside content of 5.29%;

8. Total Steviol Glycoside content of 18.04%;

9. 60.9% Rebaudioside A to total Glycoside Steviol; and

10. Over 4100 lb. of leaf per acre per year.

### BRIEF DESCRIPTION OF THE DRAWINGS

The accompanying photographs show, as nearly true as is possible in color illustrations of this type, typical flower and foliage characteristics of the ‘SW 201’ line. The plants depicted in the accompanying photographs are approximately one year old.

FIG. 1 depicts a color photograph of a fully mature ‘SW 201’ Stevia plant from roots to top of the plant near the end of the plant's first growing season.

FIG. 2 depicts a color photograph of ‘SW 201’ leaves.

FIG. 3 depicts a color photograph of an ‘SW 201’ leaf, including the shape and dimensions.

FIG. 4 depicts a color photograph of the flowers of an ‘SW 201’ Stevia plant.

### DETAILED BOTANICAL DESCRIPTION

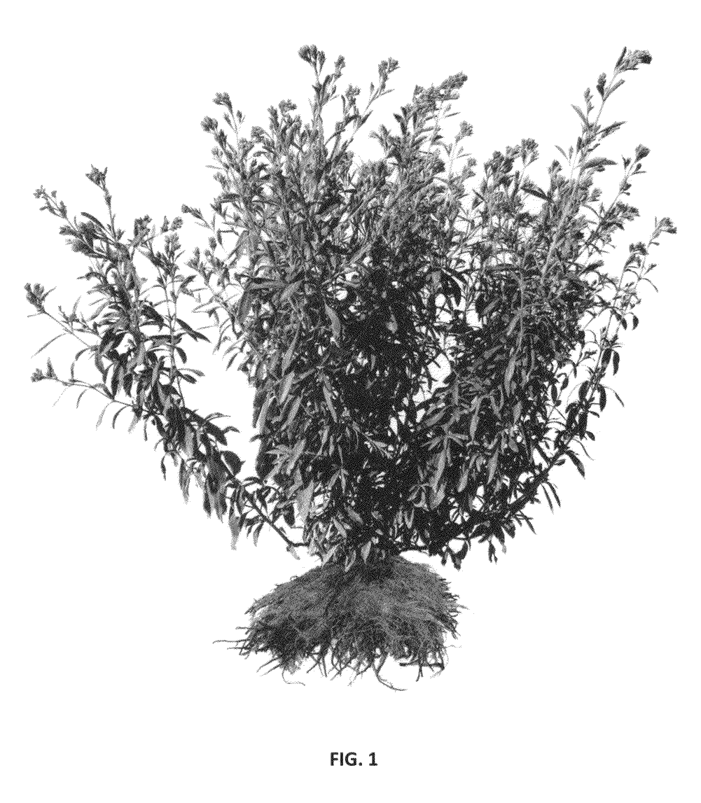

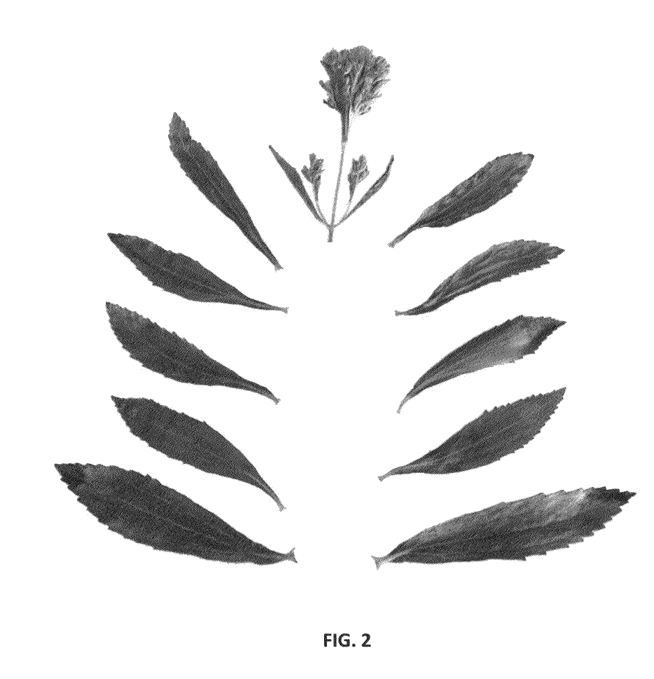

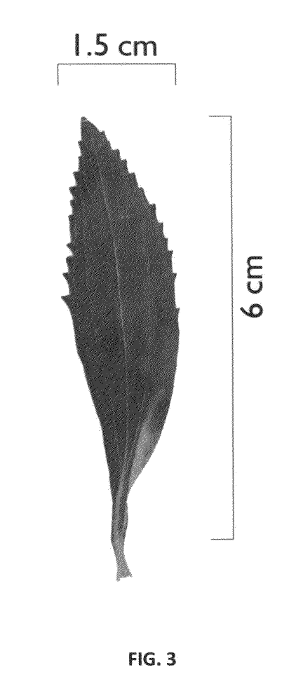

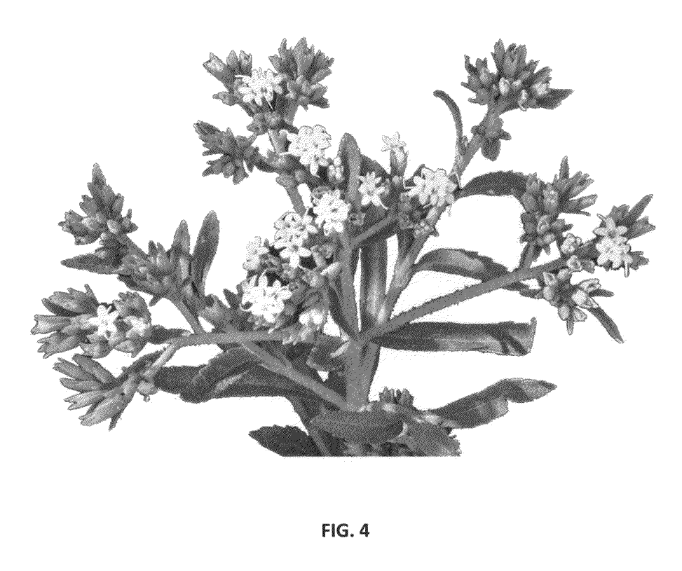

Following is a detailed botanical description of a new and distinct variety of a Stevia plant known as ‘SW 201.’ Plant observations were made on Oct. 14, 2014, on mature one year old plants grown in a field in Ontario, Oreg., USA in natural light. The ‘SW 201’ line has not been observed under all possible environmental conditions. Where characteristics, such as dimensions, sizes, colors, and other such characteristics are given, it is to be understood that such characteristics are approximations or averages and that such approximations are not intended to be limiting. It is also to be understood that the phenotype of the variety may be altered by changes with environment, such as season, temperature, light quality, light intensity, day length, cultural conditions, and the like. Color notations are based on The Royal Horticultural Society Colour Chart, The Royal Horticultural Society, London, 2007.
Botanical classification:
   Stevia rebaudiana cultivar SW 201.
Parentage: Seeds of Clinton C. Shock and stevia plants grown from the seeds in Chowchilla, Calif.
Plant (description based on FIG. 1):
   Type.—Perennial in climates with mild winter temperatures.
   Form and habit.—Multiple branching bush.
   Height (from top of soil).—90 cm.
   Width (including flowers).—75 cm.
   Number of nodes on main stem.—Varies.
   Number of basal buds.—Many.
   Harvest cycle.—Summer and fall or fall.
Leaves (description based on FIGS. 2 and 3):
   Length.—5.0 cm-10 cm.
   Width.—1.0 cm-2.0 cm.
   Arrangement.—Opposite.
   Shape.—Lanceolate.
   Apex descriptor.—Acute.
   Base descriptor.—Cuneate.
   Margin descriptor.—Serrate.
   Color, mature foliage.—Upper surface. RHS137C. Lower surface. RHS137C.
   Color, veins.—RHS 139C.
   Texture, mature foliage.—Upper surface. Slight pubescence. Lower surface. Moderate pubescence.
   Petiole length.—0.6 cm.
   Petiole diameter.—0.3 cm.
   Petiole texture.—Upper surface: Slight pubescence. Lower surface: Slight pubescence.
   Pubescence color.—Upper surface. RHS 139C. Lower surface. RHS 139C.
   Stevioside content.—5.29%.
   Rebaudioside A content.—11.17%.
   Rebaudioside A as a percentage of total steviol glycosides.—60.9%.
   Ratio of rebaudioside A to stevioside.—2.19.
   Total steviol glycosides.—18.04%.
   Leaf harvest period.—Summer and/or Fall.
Stem:
   Length (excluding inflorescence).—Varies to 1 m.
   Diameter.—Varies to 2.0 cm.
   Internode length.—Varies.
   Texture.—Pubescent.
   Color.—RHS 145B.
Flowers (description based on FIG. 4):
   Inflorescence type.—Panicle.
   Petal color.—RHS N155D.
   Petal length.—0.6cm.
   Petal width.—0.1cm.
   Bloom period.—October to November at Ontario, Oreg.
   Arrangement.—Composite.
   Shape.—Cluster.
   Fragrance.—None.
   Number of flowers per capitulum.—5.
Peduncle:
   Color.—RHS 145B.
   Length (cm).—Varies.
   Diameter.—0.15 cm.
   Aspect.—Perpendicular.
   Texture.—Pubescent.
Pedicles:
   Color.—RHS 145B.
   Length (cm).—Varies.
   Diameter.—0.03 cm.
   Aspect.—45 degrees.
   Texture.—Pubescent.
Disc florets:
   Arrangement.—Radially symmetrical.
   Shape.—Tubular.
Phyllaries:
   Quantity.—5.
   Length.—0.7 cm.
   Width.—0.15 cm.
   Shape.—Lanceolate.
   Apex.—Acute.
   Base.—Fused.
   Margin.—Overlapping.
   Color.—RHS 145B.
Reproductive organs:
   Androecium.—Stamen quantity. 5. Anther length. 0.4 cm. Anther color. RHS 199A.
   Gynoecium.—Pistil length. 0.8cm. Style color. RHS N155B. Stigma color. RHS N155B.
Fruit and seed set:
   Flowering.—Later than most stevia lines.
   Seeds.—Small black achene with pappus attached typical of Stevia rebaudiana.
Cold hardiness: Hardy to 23° F. (−5° C.) at Ontario, Oreg.
Heat tolerance: Hardy to a monthly average maximum temperature of 107-108° F. (42° C.) at Yuma, Ariz., and Indio, Calif.

We claim:

1. A new and distinct cultivar of Stevia plant named ‘SW 201’ as substantially shown and described herein.
